(19) United States
(12) Patent Application Publication
(10) Pub. No.: US 2005/0165263 A1
(43) Pub. Date: Jul. 28, 2005

(54) Catalyst for the dehydrogenation of ethylbenzene to styrene

(76) Inventors: Lucio Forni, Milano (IT); Nicola Pernicone, Milano (IT)

Correspondence Address: YOUNG & THOMPSON, 745 SOUTH 23RD STREET, 2ND FLOOR, ARLINGTON, VA, 22202, US

(21) Appl. No.: 10/510,105

(22) PCT Filed: Mar. 31, 2003
(86) PCT No.: PCT/EP03/03317

(30) Foreign Application Priority Data
Apr. 5, 2002 (IT) MI2002A000702

(51) Int. Cl. C07C 2/64; B01J 23/78

(52) U.S. Cl. 585/444; 502/328; 502/304

(57) ABSTRACT

The present invention relates to a catalyst for the dehydrogenation of ethylbenzene to styrene and to a process for the preparation thereof. The catalyst contains iron oxide, potassium oxide and calcium oxide, preferably also cerium oxide and molybdenum oxide, and is characterised by a magnesium content (expressed as MegO) lower than 0.001 %.

### STATE OF THE ART

The industrial production of styrene from ethylbenzene started in the Forties, but a large scale production started only in the Fifties, by means of a catalyst based on Fe₂O₃—Cr₂O₃—K₂CO₃, calcined at very high temperature, usually higher than 900° C. (U.S. Pat. No. 2,461,147). Due to the high calcination temperature; this catalyst, known with the trade name Shell 105, was characterised by high mechanical strength, but had low selectivity to styrene. A dramatic selectivity improvement was achieved with catalysts based on Fe₂O₃—K₂CO₃—MoO₃—CeO₂ (U.S. Pat. No. 3,904,552), which required a much lower calcination temperature (ca. 500° C.). In order to confer mechanical strength to these catalysts it was necessary to add Portland cement, which contained other elements, whose characteristics were detrimental to the catalytic process. More recently (U.S. Pat. No. 4,467,046), it has been found that the addition of a calcium compound (oxide or carbonate) to the formulation disclosed in U.S. Pat. No. 3,904,552, allowed to avoid the addition of Portland cement and its negative effects. Nevertheless, the addition of calcium oxide and carbonate renders the catalysts relatively fragile.

### DESCRIPTION OF THE INVENTION

It has now surprisingly been found that catalysts for the dehydrogenation of ethylbenzene to styrene which contain calcium oxide or carbonate are mechanically more resistant, more active and more selective when the magnesium content of the calcium oxide precursor and of the resulting catalyst is particularly low.

The present invention provides a catalyst containing iron oxide, potassium oxide and calcium oxide, preferably also cerium oxide and molybdenum oxide, characterised in that the magnesium content is lower than 0.001%. Preferably, the catalyst comprises (expressed as weight % of oxides): 50-95% iron oxide (Fe₂O₃), 5-20% potassium oxide, 0.2-14% calcium oxide, 0.5-20% cerium oxide and 0.2-8% molybdenum oxide. A more preferred composition comprises: 60-85% iron oxide (Fe₂O₃), 5-15% potassium oxide, 0.5-2% calcium oxide, 5-15% cerium oxide, 0.5-2% molybdenum oxide. A most preferred composition comprises: 75-80% iron oxide (Fe₂O₃), 8-12% potassium oxide, 0.7-1.4% calcium oxide, 8-12% cerium oxide, 0.7-1.4% molybdenum oxide.

The catalyst of the invention can be prepared from iron, calcium, potassium, cerium and molybdenum salts, hydroxides or variously hydrated oxides, easily available on the market, the magnesium content of said compounds being such that the final catalyst contains less than 0.001% by weight of magnesium (expressed as magnesium oxide).

A further object of the present invention is therefore a process for the preparation of the catalyst comprising the following steps:
pre-mixing iron, calcium, potassium, cerium and molybdenum salts, hydroxides or variously hydrated oxides having a magnesium content such that the final catalyst contains less than 0.001% by weight of magnesium (expressed as magnesium oxide);
adding a solution or a suspension of a binder-lubricating agent so as to obtain a paste;
extruding a paste in cylindrical particles of diameter and length ranging from 2 to 6 mm;
submitting said cylindrical particles to drying and calcination characterised in that calcination is carried out for a time range from 30 minutes to 10 hours, preferably from 1 to 4 hours, at a temperature ranging from 950 to 990° C., preferably from 960 to 980° C. and more
preferably from 965 to 975° C.

Pre-mixing can be carried out by means of conventional techniques, such as dry or wet milling, e.g. in a ball mill or in another suitable apparatus and added with a proper amount of a solution or suspension of a binder-lubricating agent, such as stearic acid, carboxymethyl-cellulose, polyethylene glycol, glycerol, starch of various origin and similar compounds, so as to form a paste of the proper consistency, suitable for the extrusion in cylindrical particles. Before calcination, the extruded cylinders are dried at a temperature between 50 and 120° C., preferably between 60 and 100° C.

The catalyst of the invention can be conveniently used for the dehydrogenation of ethylbenzene to styrene by passing a flow of ethylbenzene and water vapour through a bed of catalyst particles, with a “steam/oil” (S/O) water/ethylbenzene weight ratio between 2.5 and 1.0, preferably lower than 2. It is well known that the lower the S/O ratio, the lower the energy consumption of the process, but the higher the risk of catalyst deactivation, due to deposition of carbonaceous compounds. A nitrogen flow is added to these two reactants, so as to give a volumetric dilution ratio (RD) of the gaseous flows (ethylbenzene+water)/(ethylbenzene+water+nitrogen) between 0.1 and 1, preferably between 0.4 and 0.6.

The present invention will be now illustrated in further detail by means of some examples.

### EXAMPLES

### Examples 1-5

A mixture of finely powdered iron oxide, cerium carbonate, calcium carbonate, potassium carbonate and potassium molybdate, in proper weight ratios, (said mixture containing less than 0.001% by weight of magnesium, expressed as magnesium oxide) has been dry milled for 4 hours in a corundum ball mill, so as to give a final catalyst with the following weight % composition: Fe₂O₃ 78, CeO₂ 10, CaO 1, K₂O 10, MoO₃ 1. A 5% by weight aqueous suspension of carboxymethyl-cellulose as binder-lubricating agent has been then added to the mixture so as to form a paste having a consistency suitable for extrusion through a die with holes 3 mm in diameter. The extrudates have been dried overnight at 80° C. in an oven and divided in 5 portions. The latter have been calcined separately in flowing air, with a temperature ramp of 1° C./min, up to 900, 950, 970, 990 and 1000° C., respectively. The final calcination temperature has been maintained for 3 hours and then the samples have been allowed to cool down to room temperature. The cylindrical particles thus obtained (3×3 mm in size), have been subjected to the crushing strength essay, as described in the literature (see e.g. J. T. Richardson, “Principal of catalyst development”, Plenum Press, New York 1989) and then crushed and sieved, and the 40-60 mesh fraction (samples IC1-90, IC1-95, IC1-97, IC1-99 and IC1-100) has been recovered.

Every sample has been tested by loading 1 g of catalyst in a continuous tubular laboratory reactor, made of Incoloy 800 alloy (internal diameter 9 mm, with an axial thermo well of 1.6 mm external diameter). A nitrogen flow has been then fed to the reactor and the temperature was raised by 3.17° C./min up to 400° C., then by 1.75° C./min up to 610° C., then maintained. The feeding of water and of ethylbenzene was started at 300° C. and at 550° C., respectively. The space velocity (LHSV) was 0.7 cm³ of ethylbenzene/(hour×cm³ of catalyst bed), the S/O ratio was 2 and the RD ratio was 0.5.

The samples of reactor effluent for the determination of the activity and selectivity of the catalyst, have been collected by means of traps, cooled to −40° C. by a cryostat. The conversion of ethylbenzene and the selectivity to styrene have been determined at 48 hours-on-stream, by gas-chromatographic analysis of the effluent samples collected in the traps. The results of the mechanical strength and catalytic activity assays are reported in table 1.

### Example 6

A sample of catalyst, referred to as IC3-97a, consisting of: Fe₂O₃ 78%, CeO₂ 10%, CaO 1%, K₂O 10%, MoO₃ 1%, prepared by employing as CaO precursor a commercial calcium carbonate containing 0.5% by weight of magnesium carbonate, and operating as described in examples 1-5, has been calcined at 970° C. and tested under the conditions described above. The results are reported in Table 1.

### Example 7

A sample of catalyst, referred to as IC3-97b, consisting of: Fe₂O₃ 77%, CeO₂ 10%, CaO 1%, K₂O 10%, MoO₃ 1%, MgO 1%, has been calcined at 970° C. and tested under the conditions described above. The results are reported in Table 1.

### Example 8

A sample of catalyst IC1-97, tested under the same conditions as those of examples 1-5, but with a S/O ratio of 1.5, gave the results reported in Table 1. These results remained practically unaltered after 900 hours on stream.

TABLE 1

| | | Conv. of Ethylbenzene | Selectivity to | Mech. |
|---|---|---|---|---|
| Catalyst | S/O | Mol % | Styrene Mol % | Strength |
| IC1-90 | 2 | 69.26 | 93.06 | fair |
| IC1-95 | 2 | 75.30 | 93.37 | good |
| IC1-97 | 2 | 85.53 | 94.04 | excellent |
| IC1-99 | 2 | 77.40 | 94.01 | excellent |
| IC1-100 | 2 | 65.35 | 94.68 | excellent |
| IC3-97a | 2 | 73.42 | 94.58 | fair |
| IC3-97b | 2 | 66.04 | 94.60 | insufficient |
| IC1-97 | 1.5 | 84.50 | 94.02 | excellent |

The above examples demonstrate that other conditions being the same (calcination temperature, nature of precursors and preparation procedure) the presence of magnesium noticeably lowers both mechanical strength and conversion, while insignificantly affects selectivity (compare catalyst IC1-97, virtually magnesium-free, with IC3-97a, containing significant traces of MgO, and with IC3-97b, containing 1% of MgO).

For identical compositions, the higher the calcination temperature, the higher the mechanical strength of the catalyst (compare catalysts IC1-90, IC1-95, IC1-97, IC1-99 and IC1-100). When the calcination temperature exceeds 970° C., a considerable decrease of activity can be observed, the decrease being remarkable when the calcination temperature exceeds 990° C. (compare catalysts IC1-97, IC1-99 and IC1-100).

1. A catalyst for the dehydrogenation of ethylbenzene to styrene, containing iron oxide, potassium oxide and calcium oxide, characterised in that the magnesium content expressed as magnesium oxide is lower than 0.001%.

2. A catalyst according to claim 1 characterised in that it also contains cerium oxide and molybdenum oxide.

3. A catalyst according to claim 2, comprising: 50-95% Fe₂O₃, 5-20% K₂O, 0.2-14% CaO, 0.5-20% CeO₂, 0.2-8 MoO₃.

4. A catalyst according to claim 3, comprising: 60-85% Fe₂O₃, 5-15% K₂O, 0.5-2% CaO, 5-15% CeO₂, 0.5-2 MoO₃.

5. A catalyst according to claim 4, comprising: 75-80% Fe₂O₃, 8-12% K₂O, 0.7-1.4% CaO, 8-12% CeO₂, 0.7-1.4 MoO₃.

6. A catalyst according to claim 5, comprising: 78% Fe₂O₃, 10% K₂O, 1% CaO, 10% CeO₂, 1% MoO₃.

7. A process for the preparation of the catalyst of claim 1, comprising:
pre-mixing iron, calcium, potassium, cerium and molybdenum salts, hydroxides or variously hydrated oxides;
adding a solution or a suspension of a binder-lubricating agent so as to obtain a paste;
extruding the paste in cylindrical particles of diameter and length ranging from 2 to 6 mm;
submitting said cylindrical particles to drying and calcination
characterised in that the catalyst contains less that 0.001% of magnesium, expressed as magnesium oxide.

8. A process according to claim 7, characterised in that the final calcination of the catalyst is carried out in a temperature range from 950 to 990° C.

9. A process according to claim 8, characterised in that the final calcination is carried out in a temperature range from 960 to 980° C.

10. A process according to claim 9, characterised in that the final calcination is carried out in a temperature range from 965 to 975° C.

11. A process according,to claim 10,
characterised in that the final calcination is carried out at 970° C.

12. A process according to claim 6, characterised in that the final calcination time ranges from 30 min to 10 hours.

13. A process according to claim 12,
characterised in that the final calcination time ranges from 1 hour to 4 hours.

14. A catalyst obtainable according to the process of claim 7.

15. A process for the dehydrogenation of ethylbenzene to styrene which comprises passing a flow of ethylbenzene and water vapour through a bed of particles of the catalyst of claim 1.

16. A process according to claim 15,
characterised in that the water/ethylbenzene ratio is lower than 2.

17. A process according to claim 16,
characterised in that the water/ethylbenzene ratio not higher than 1.5.
